(19) United States
(12) Patent Application Publication
(10) Pub. No.: US 2012/0003300 A1
(43) Pub. Date: Jan. 5, 2012

(54) Composition Comprising Vascular Endothelial Growth Factor (VEGF) for the Treatment of Hair Loss

(75) Inventors: Elliot Isaacs, London (GB); Toby Cobbledick, London (GB)

(73) Assignee: Pangaea Laboratories Ltd

(21) Appl. No.: 13/173,894

(22) Filed: Jun. 30, 2011

(30) Foreign Application Priority Data
Jun. 30, 2010 (GB) 1010970.0
Oct. 19, 2010 (GB) 1017674.1
Feb. 3, 2011 (GB) 1101877.7

(51) Int. Cl. A61K 38/18; A61K 36/82; A61K 36/889; A61K 36/48; A61K 36/15; A61K 36/11; A61K 35/32; A61K 9/127; A61P 17/14; A61P 31/10; A61P 31/00; A61P 29/00; A61P 33/00; A61P 17/18; A61K 38/44

(52) U.S. Cl. 424/450; 514/8.1; 424/94.4; 424/729; 424/727; 424/757; 424/59; 424/60; 424/62; 424/65; 424/770; 424/725; 424/548

(57) ABSTRACT

A composition to treat hair loss and enhance hair growth and condition. The composition comprises: i) one or more of VEGF, a VEGF biomimetic peptide, and/or a VEGFR2 receptor agonist; ii) ciclopirox olamine; and iii) a pharmaceutically acceptable carrier.

The present invention relates to the use of ciclopirox olamine, in combination with vascular endothelial growth factor (VEGF), to improve hair growth and condition.

The process of new hair growth, whether as part of the natural hair cycle or as a result of a treatment to encourage hair growth, relies on numerous cross-talking signal pathways to bring about the processes necessary for hair growth. These principal processes are: cell proliferation of the dermal papilla, cell migration to form the appropriate structures, and angiogenesis to form blood supply routes to the new hair follicle.

The three steps are also vital for healthy skin condition. In normal skin and normal hair growth cycling, these stages take place without any pharmaceutical or cosmetic intervention. However, in people with various forms of alopecia, hair loss, or a slowing of hair growth, one or more of these processes might not happen at a normal rate, and can stop altogether. Similarly, these processes often occur at a reduced rate in UV damaged skin and scar tissue.

Vascular endothelial growth factor (VEGF) is a well-documented signal to activate these processes via the VEGFR2 receptor. VEGF leads to downregulation of Bad, TGF-β1 and caspase expression through the Akt/PKB and calcium ion dependent pathways, thereby bringing about the end of apoptosis and the telogen phase. Secondly it acts via the MAPKinase pathway to increase cell proliferation. Via the same Akt/PKB and calcium ion dependent pathways, VEGF also stimulates nitric oxide (NO) production and cell migration. Interestingly, all of these pathways, excepting the Akt/PKB pathway, are calcium ion dependent.

Without doubt the key to maintaining hair shaft growth is a sufficient supply of oxygen and essential amino acids. This requires blood. As has been shown by repeated experiments, increasing blood flow and blood vessel formation to hair follicles improves and maintains hair growth, without this hair growth will cease.

VEGF is the cytokine solely responsible for blood vessel formation. Via the VEGFR2 receptor, VEGF stimulates vascular cells to proliferate to extend the blood vessel, migrate and organise to form the vessel organ. This rapidly extends new vessels into areas that require blood supply. VEGF also stimulates eNOS to create nitric oxide, stimulating blood vessel and cell membrane permeability for the efficient transfer of nutrients. VEGF is particularly useful because VEGF upregulation and many related pathways are stimulated by hypoxia, so it acts where it is needed (Hoeben A., et al., (2004) Pharmacology Review, 56 p. 549-8).

Dihydrotestosterone has long been studied as a major cause of hair loss. It is a hormonal signal which penetrates the follicle, and causes down-regulation of Bcl-2 leading to apoptosis. Bcl-2 interacts with Bax and Bad genes to prevent apoptosis, and so lowering the concentration of Bcl-2 allows Bax and Bad to promote apoptosis through the same pathway (Hoeben A., et al., (2004) Pharmacology Review, 56 p. 549-8).

VEGF blocks Bad conversion, maintaining the pre-apoptotic state (Manning B. D., Cantley L. C., (2007) Cell, 129(7) p. 1261-1274). This means that Bcl-2 only has Bax to interact with, and so prevents apoptosis at lower concentrations. VEGF also leads to the down-regulation of caspase 9 (Manning B. D., Cantley L. C., (2007) Cell, 129(7) p. 1261-1274), reducing a different apoptotic pathway. VEGF has a clear role in preventing early apoptosis via these two pathways, and the prevention of hypoxia and oxidative stress. This will lead to the maintenance of Anagen for longer.

Minoxidil is the treatment of choice for most physicians when treating hair loss. It is surprising then that several modes of action have been theorised, but none of them proven.

The principle mode of action for minoxidil is thought to be the donation of nitric oxide. This gaseous signalling molecule is well known to cause vasodilation and improve circulation. Nitric oxide signals are degraded rapidly by free radicals, therefore many treatments utilise antioxidants to prolong the signal life. A notable antioxidant is superoxide dismutase. As an enzyme with a high turnover rate, superoxide dismutase removes may reactive oxygen species, and effectively reduces nitric oxide breakdown.

A credible, newer theory is that minoxidil's effects, opening the Na⁺/K⁺ATPase channel, promote hair growth. This effect has been shown by the classification of two channel subtypes in the follicle, one of which is opened by minoxidil. When opened by a different specific channel opener, hair growth was improved while, when a channel inhibitor was used, the growth effect was prevented (Shorter K., et al., (2008) FASEB Journal, 22(6) p. 1725-36).

The Na⁺/K⁺ ATPase channel has another function, regulating Ca²⁺ ion levels. Permanently opening the channels causes the levels of Ca²⁺ ions to stabilise. As the proliferative and eNOS stimulating effects of VEGF are Ca²⁺ mediated, there is evidence that maintenance of Ca²⁺ ion levels is necessary for a VEGFR2 signal transmission to be effective. Therefore minoxidil may make a VEGF signal more efficient and increase the intracellular effects of VEGF.

An interesting corollary to the possibility of minoxidil acting with or via VEGF is the fact that minoxidil upregulates VEGF expression in anagen dermal papilla cells. This upregulation ensures adequate vascularisation of the follicle through the anagen phase and is likely to explain at least part of the mode of action of minoxidil (Lachgar, et al., (1998) British Journal of Dermatology, 1998. 138(3) p. 407-411).

Prostaglandins have been another widely researched treatment option. Research suggests that prostaglandins are active in the very early stages of anagen, possibly even at the initiation step, as suggested by the new eyelash growth in several clinical trials (Johnstone M. A., Albert D. M., (2002) Survey of Ophthalmology, 47(1): p. S185-202). The prostaglandin system is complex, made from a large number of different prostaglandins, and is still not fully researched.

VEGF has been shown to induce prostaglandin I(2) production in epithelial cells. Prostaglandin I(2) is unlikely to stimulate new hair growth, however prostaglandin I(2) receptors have been found to be specifically expressed in hair cuticle layer, suggesting an important role for hair matrix cell differentiation to form the outer hair cuticle (Colombe L., Michelet J. F., Bernard B. A., (2008) Experimental Dermatology, 17(1) p. 63-72). This outer layer is essential for terminal hair formation.

This may also explain the necessity for VEGF upregulation in the early anagen stage mentioned earlier.

Widely regarded as the most successful treatment for alopecia areata, diphencyprone is another treatment with no definite mechanism. As a potent allergen, topical application of diphencyprone as an immunotherapeutic agent stimulates a response and leads to normalisation of hair growth (Happle R., (2002) Archives of Dermatology, 138 p. 112-113).

Recent work shows this “response” is threefold. Firstly, the ratio of CD4/CD8 cells is known to differ significantly in alopecia areata patients. Diphencyprone stimulates a normalisation of this ratio to one approaching normal scalp tissue. Diphencyprone also upregulates the expression of survivin, thereby helping to preventing the premature apoptosis symptomatic of alopecia areata patients. Lastly, it upregulates the expression of VEGF in hair follicle keratinocytes, maintaining nutrient and oxygen supply (Simonetti O., et al., (2004) British Journal of Dermatology, 150(5) p. 940-948). VEGF also has an anti-apoptotic role, downregulating Casp9 and Bad genes which are key to follicle apoptosis (Manning B. D., Cantley L. C., (2007) Cell, 129(7) p. 1261-1274).

Whilst alopecia areata pathenogenesis is still unknown, VEGF explains part of the success of the most successful treatment to date.

In summary, it is well known that minoxidil and anti-dihydrotestosterone treatments are effective against androgenetic alopecia, and diphencyprone is a useful treatment for alopecia areata. Cytokines, particularly VEGF, provide an alternative to these treatments, or a supporting role to all of these known therapeutic options.

There are clearly diverse possibilities for VEGF, either as an independent treatment or to supplement minoxidil for androgenetic alopecia, or diphencyprone for alopecia areata. As the role in stimulating vascularisation is clear, it is also likely that a solution containing VEGF would help graft survival and improve wound healing times after surgery.

It is also conceivable that VEGF could be added alone, or with other growth factors to an autologous growth factor treatment such as Platelet Rich Plasma therapy.

However, VEGF is a large molecule of between 20 and 40 kDa depending on the source and, when applied topically to normal human epidermis, penetration is low. Research into molecule penetration has shown that penetration via the hair shaft is possible. Penetration through the thinner epidermis around pores and hair shafts may also be sufficient to allow a useful amount of VEGF to be active.

An alternative way to enhance penetration of larger molecules through the skin, particularly the scalp, is to use a microneedle array. The microneedles create quickly healing channels through the stratum corneum, allowing over five times penetration of many molecules, including proteins far larger than VEGF (Verbaan, F. J., et al., (2007) Journal of Controlled Release, 117(2) p. 238-245).

Therefore, there is a need to find a way to improve the penetration of VEGF and to enhance the action of VEGF at its target to stimulate and enhance hair growth.

In its search to improve the topical effects of VEGF on hair loss and growth, the Applicant has found that a combination of VEGF with an iron chelator provides a suitable solution. Iron chelators essentially bind iron to make the metal ion inert within the body. Known iron chelators include ethylenediaminetetraacetic acid (EDTA), ferrous bisglycinate, Desferoxamine, 2,2′-dipyridyl 1,10-phenanthroline, 2,2′-dipyridylamine 2-furildioxime, N-(4-pyridoxy/methylene)-1-serine and Kojic acid. However, the Applicant has found that ciclopirox, particularly ciclopirox olamine, is especially suitable for the present purpose.

Ciclopirox, or 6-cyclohexyl-1-hydroy-4-methyl-1,2-dihydropytidin-2-one, is an active antifungal ingredient of the family of hydroxyl pyridones which is finding increasingly greater use in the treatment of seborrheic dermatitis and dandruff due to its chelating capacity of ferric ions (EP 2275104). Ciclopirox olamine is also known to induce hypoxia-inducible factor 1-alpha (HIF-1alpha), VEGF expression and angiogenesis in the context of wound healing (Linden T., et al., (2003) FASEB Journal, 17 p. 761-763).

Accordingly, the present invention encompasses the use of VEGF, in combination with ciclopirox olamine, to treat hair loss and enhance hair growth and condition.

Expressed in another way, the present invention resides in a composition to treat hair loss and enhance hair growth and condition, the composition comprising: i) one or more of VEGF, a VEGF biomimetic peptide, and/or a VEGFR2 receptor agonist; ii) ciclopirox olamine; and iii) a pharmaceutically acceptable carrier.

In the composition of the invention, the VEGF, a VEGF biomimetic, and/or a VEGFR2 receptor agonist may be one or more of: Copper Ascorbyl Phosphate Succinoyl Tripeptide-34; the peptide: Glycine Histadine Lysine-Cu; the peptide: R-Glycine Histadine Lysine-Cu, where R is any amino acid or peptide chain; Octapeptide-2; Decapeptide-8; Synthetic Human VEGF; Recombinant Human VEGF; Human VEGF from any human source.

The composition may comprise between 0.0001% and 50% of ciclopirox olamine. Ideally, ciclopirox olamine is present in the composition in an amount of between about 0.03% and about 0.50%.

The composition may comprise between 0.00001% and 45% of VEGF, VEGF biomimetic, and/or VEGFR2 receptor agonist. Ideally, VEGF, VEGF biomimetic, and/or VEGFR2 receptor agonist is present in an amount of between about 0.03% and about 0.12% or between about 1 and 10 ppm.

As mentioned above, ciclopirox olamine is known to cause an upregulation of VEGF production, with this increase causing a corresponding increase in the downstream effects of VEGF discussed previously. Ciclopirox olamine may act as a sodium and potassium ion channel antagonist which, in turn, may increase cellular calcium ion concentration. This increase in calcium will undoubtedly improve the intracellular signalling potential of the calcium dependent pathways, thereby having a potentiating affect on intracellular signals.

Whilst the effects of ciclopirox olamine alone on hair growth may be statistically significant, the results do not make enough of a difference for visual evaluation in in vivo test subjects and thus for any effect to be appreciated by a patient. Therefore, for use in pharmaceutical and cosmetic preparations, ciclopirox olamine must be used with an additional compound to enhance the result and achieve a difference that is visible and appreciable to a patient.

Human VEGF has not yet been widely legalised for cosmetic or pharmaceutical use. However, it is available as a variety of synthetic and recombinant forms. More widely available are VEGFR2 receptor agonists, and several different biomimetic peptides, that may act as VEGFR2 receptor agonists or act in a similar way downstream of the VEGFR2 receptor. The agonists stimulate and/or potentiate the calcium ion dependent, MAPKinase, or Akt/PKB pathways, among other pathways naturally stimulated by VEGF. Any of these are considered suitable for inclusion in a composition with ciclopirox olamine to improve the effects of either component.

This synergy between VEGF, VEGFR2 agonists and/or VEGF biomimetic peptides and ciclopirox olamine occurs in two ways. Firstly ciclopirox olamine increases cellular calcium ion concentration. In turn, this potentiates the intracellular signals stimulated not only by the VEGF that ciclopirox olamine produces, but also any additional receptor stimulation by VEGF, VEGFR2 agonists and/or VEGF biomimetic peptides included in a composition. Secondly, VEGF, VEGFR2 agonists and/or VEGF biomimetic peptides stimulate the VEGFR2 receptor in any set of conditions. This is not the case for the action of ciclopirox olamine, which up-regulates VEGF production more significantly in hypoxic conditions. Therefore, in well oxygenated areas, ciclopirox olamine may not have a significant upregulating effect on VEGF and the presence of VEGF, VEGFR2 agonists and/or VEGF biomimetic peptides in the composition will ensure reliable results in all areas of applicant to the skin.

Additionally, ciclopirox olamine has limited water solubility. Whilst the considered pharmaceutical and cosmetic compositions may be based in oils, water, alcohols, propylene glycol or many other media, water based solutions have a variety of advantages. However, a water based solution of ciclopirox olamine would not create enough VEGF via up-regulation to have a significant effect and so the addition of VEGF, VEGFR2 agonists and/or VEGF biomimetic peptides allows for significant results in water based solutions.

Incorporation of ciclopirox olamine may have another benefit as it is known to be an anti-fungal agent and so may be used to prevent and cure dandruff and infections by Malessezia furfur and other fungi. As infections may also be treated by electromagnetic radiation—either UV or a cold-beam laser—it is considered that the composition of the invention may be used with sources of electromagnetic radiation, particularly UV, blue and/or red visible light. These could be emitted by light sources such as LEDs or laser emitters. Laser emitters are also thought to have a positive effect on skin and hair condition and so the possibility of using the composition of the invention with regular laser therapy is also considered.

As the cell migration action of VEGF partially relies on nitric oxide production, a favourable embodiment may additionally comprise one or more superoxide dismutases or superoxide dismutase mimetics. Alternatively, or additionally, it may comprise one or more catalases or catalase mimetics. These molecules act together to remove reactive oxygen and nitrogen species, prolonging the duration of the nitric oxide signal. Possible superoxide dismutases are any of, but not limited to, the following: Cu/Zn Superoxide Dismutase, Mn Superoxide Dismutase, Lipochroman-6, EUK-134, copper (II) 3,5-diisopropylsalicylate, copper (II) 3,5-dibromosalicylate, copper (II) 3,5-ditertiarybutylsalycilate. Possible antioxidants are either of, but not limited to, green tea extract and vitamin C or derivative thereof. Possible catalases or catalase mimetics are any of, but not limited to, copper PCA, zinc PCA, Catalase.

An alternative or additional embodiment may further comprise an antioxidant, to prevent free radical degradation of nitric oxide, or a UV protective agent to reduce UV damage and free radical formation. Examples of suitable antioxidants include EUK-134, copper (II) 3,5-diisopropylsalicylate, copper (II) 3,5-dibromosalicylate, copper (II) 3,5-ditertiarybutylsalycilate, green tea extract and vitamin C or derivative thereof.

Embodiments including UV protective agents have the additional benefit of preventing further skin blemishes such as hyperpigmentation, to which alopecia sufferers and those with scar tissue may be more susceptible. Various UV protective agents are considered within the scope of the invention, including but not limited to any of the following: propanediol, quaternium-95, polyacrylate-15, titanium dioxide, zinc oxide, ethylhexyl methoxycinnamate, butyl methoxydibenzoylmethane, octyl methoxycinnamate, menthyl anthranilate, homosalate, benzophenone-3 and benzophenone-4.

Similarly, other forms of increasing nitric oxide may have a beneficial effect. Therefore, the composition may additionally comprise one or more natural or synthetic nitric oxide donators or mimetics. Embodiments including nitric oxide synthases, nitric oxide donators, and nitric oxide mimetics such as any of eNOS, iNOS, minoxidil, 2,2,6,6-tetramethylpiperidine-N-oxyl (TEMPO), molsidomine and S-nitrosoglutathione are also considered.

The anti-apoptotic action of VEGF, VEGFR agonists and VEGF biomimetic peptides occurs largely downstream of extracellular apoptotic signals. Whilst this has the benefit of being more specific, targeting the apoptotic pathway prior to the intracellular signals does increase the possible amplitude of the effect. Due to this, a beneficial embodiment may include one or more 5-alpha reductase inhibitors for reducing dihydrotestosterone levels such as zinc PCA, finasteride, dutasteride, turosteride, bexlosteride, izonsteride, FCE 28260, SKF 105,111, saw palmetto extract, green tea extract and hydrolysed lupine extract, and/or another means for reducing the effects of dihydrotestosterone such as caffeine.

Stimulation of VEGFR2 receptors and increasing signal transduction may increase circulation and, therefore, have a beneficial effect as part of many cosmetic preparations when included with other cosmetically active agents. Cosmetically active agents are defined herein as compounds (natural or synthetic) that have a cosmetic or therapeutic effect on the skin, hair or nails including but not limited to lightening agents, darkening agents, anti-acne agents, shine control agents, anti-microbial agents, anti-inflammatory agents, anti-mycotic agents, anti-parasite agents, external analgesic, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning. Of particular interest are curcumin, taurine, plant sterols, pine bark extract, red tea, white tea, horsetail extract, marine cartilage, kieslerde, melatonin and mimetics, copper peptides, other growth factors and growth factor mimetics, spironolactone, β-glucan, vitamins C, A, E, B, F, H, K (and derivatives), bacterial filtrates, glucosamine sulphate, or any combination of these.

Ciclopirox olamine readily penetrates skin, hair, and nail layers. However VEGF, VEGFR2 agonists and VEGF biomimetic peptides will penetrate somewhat more slowly. To improve this performance, VEGF, VEGFR2 agonists and/or VEGF biomimetic peptides may be included in the composition in an encapsulated form, such as encapsulation in liposomes or nanosomes, to increase penetration, or the preparation may additionally comprise a penetration enhancing ingredient such as salicylic acid. Alternatively or additionally ciclopirox olamine penetration may be reduced to equate to that of VEGF, VEGFR2 agonists and/or VEGF biomimetic peptides, for example by encapsulating ciclopirox olamine in a large micelle. Another alternative method for altering the absorption rate of the preparation is to include microneedling, wherein the composition is applied topically as part of a procedure including an array of microneedles.

The vasodilatory effects of VEGF pathway stimulation may also be useful as part of growth factor therapies. Autologous growth factor therapies are becoming increasingly popular for alopecia treatment, skincare and surgical purposes. Any of the embodiments discussed previously would have beneficial effects when applied as part of an autologous growth factor treatment. Non-autologous growth factors are currently not widely legalised and used, but any of the embodiments discussed previously may be beneficial as part of these potential future treatments. A specific biomimetic peptide and recombinant or synthetic sources of VEGF are considered in detail herein. However, any biomimetic peptide fulfilling this function may also be considered in addition or as an alternative to either of those described herein.

In one aspect, the invention provides a cosmetic or pharmaceutical composition comprising ciclopirox olamine and one or more of VEGF, a VEGF biomimetic, and/or a VEGFR2 receptor agonist in a physiologically acceptable medium.

Ciclopirox olamine is included to stimulate VEGF production. Ciclopirox olamine may increase intracellular VEGF signal transduction and may also act as an antifungal agent. It may also act as a sodium/potassium ion channel antagonist.

The preparation may be for use with a microneedle array. In another aspect, the invention provides a microneedle array including the preparation described herein. In another aspect, the invention provides a kit for applying the preparation, in which the kit comprises an array of microneedles and the preparation.

The preparation may be for use as part of an autologous growth factor therapy.

In a further aspect, the invention provides a method of treating or preventing hair loss, the method comprising topical application of a composition as described herein. The term “topical” as used herein means applied to the skin or scalp.

In a yet further aspect, the invention provides a method of maintaining or improving hair growth, which comprises topical application of a composition as described herein.

In a different aspect, the invention provides a method of improving skin blemishes, such as hyperpigmentation and scarring, which comprises topical application of a composition as described herein.

In a further different aspect, the invention provides a method of treating or preventing damage to skin cells associated with accumulation of reactive oxygen or nitrogen species such as free radicals, which comprises topical application of a composition as described herein.

The treatment of hair loss as used in the context of the present invention encompasses the treatment of hair loss by the surgical transplantation of hair and skin incorporating hair follicles. Thus, in another aspect, the invention provides a method of using a composition as described herein, to prepare possible implantation sites before hair transplant surgery.

In yet another aspect, the invention provides a method of using a composition as described herein, to soak follicular grafts before implantation as part of hair transplant surgery.

In an additional aspect, the invention provides a method of using a composition as described herein, after implantation of hair follicles to improve their survival rates and/or to improve wound healing times.

Finally, in another different aspect the invention provides a method of using a composition as described herein, to prepare grafts for removal prior to hair transplant surgery.

The use of the composition described above may be topical application and/or injection, or in other ways.

It is possible to utilise the angiogenic properties as well as the hair growth inducing properties of the discussed VEGF containing solution before, during or after surgery. This may be hair transplant surgery, and the solution may be used to soak follicular grafts before implantation, or used after implantation to improve survival rates and wound healing times. The composition may be beneficial before surgery to prepare grafts for removal, or prepare possible implantation sites.

The compositions of the present invention may be in the form of oil emulsion in water or water in oil, including an oily phase and an aqueous phase, or in the form of gel, spray, shampoo, solution for example for tissues or water or hydroalcoholic-based lotion or pressurised foam.

In the oil emulsions or in water or water in oil, the oily phase may contain branched or non-branched hydrocarbons such as oil of Vaseline®, paraffin, squalene, polyisobutylene, hydrogenated polydecene; silicon oils, for example dimethicone, phenyl trimethicone, cyclomethicone and dimethicone; fatty acids selected from saturated fatty acids and partially unsaturated which have between eight and twenty atoms of carbon, for example lauric, myristic, palmitic, stearic, isostearic, oleic, linoleic, eicosanoic, docosanoic, eruic; C₁-C₂₀ alkyl and alkenyl esters of fatty acids saturated and partially unsaturated which have between ten and thirty atoms of carbon, for example decyl oleate, isodecyl oleate, dioctyl maleate, isopropyl palmitate, isoesyl palmitate, ethylesyl palmitate, lauryl lactate, myristyl lactate, cetyl lactate, isostearyl neopentanoate, ethylesyl isostearate, myristyl myristate, esyl laurate, cetyl palmitate, isopropyl palmitate, hexadecyl stearate, decyl stearate, isopropyl isostearate, diisopropyl adipate, diisoesyl adipate, diesyldecyl adipate, isocetul stearoyl stearate, C₁₂-C₁₅ alkyl lactate, cetearyl isononanoate; glycerin and mixtures thereof.

The oily phase may further contain cetylic alcohol, stearylic alcohol, glyceryl stearate, polysorbates, esters or ethers of fatty acids from C₁₂-C₁₈ with polyethylene glycols.

The oily phase my also contain esters or fatty acids between C12 and C20 glycerol and polyglycerol, vitamins such as vitamin E and ureides such as allantoin.

Components of the aqueous phase may include water, alcohols such as denatured ethyl alcohol or isopropyl alcohol, organic salts such as sodium citrate, potassium sodium tartrate, potassium, sorbate, sodium benzoate and EDTA; inorganic salts such as sodium chloride, calcium chloride, sodium metabisulphite, magnesium sulphate; buffering agents, for example sodium hydroxide, sodium bicarbonate, sodium hydrogen phosphate and mixtures thereof; organic acids such as salicylic or citric acid; preservatives including parabens such as methylparaben, phenoxyethanol, chlorhexidine, chlorphenesin, imidazolidinyl urea, derivatives of glycine, phenoxyethanol, sodium benzoate, benzoic acid, tromethamine; essential oils for example eucalyptus, menthol, thyme, cinnamon, geranium, sea salt; amino acids including arginine, cysteine, methionine betaine and lysine and the their derivatives; jellying agents including derivatives of cellulose, alginates, carrageenan, derivatives of guar gum, polymers of acrylic acids, such as polyacrylates, methylacrylates, carbomer; vitamins such as, for example, niacinamide, ascorbic acid or derivatives of ascorbic acid.

The aqueous phase may further comprise glycerol, propylene glycol, butylene glycol, ethyl alcohol, isopropyl alcohol, polyalcohols, amino alcohols such as methanolamine, saccharide components including beta-glucan, derivatives of amide and cellulose.

The water used in the composition of the present invention may be deionised or mineral water. Ideally the water content of the composition is relative to the total content of the other components used so that the total weight of the composition is equal to 100% by weight of the composition. The composition ideally comprises between about 60% to about 90% water.

In a further embodiment, the present invention resides in a method of autologous growth factor therapy in the treatment of alopecia comprising use of a composition comprising: i) one or more of VEGF, a VEGF biomimetic peptide, and/or a VEGFR2 receptor agonist; ii) ciclopirox olamine; and iii) a pharmaceutically acceptable carrier.

In a yet further embodiment, the present invention encompasses a method for the prevention or treatment of hair loss, for improving hair growth, for improving skin blemishes, or for the prevention or treatment of damage to skin cells associated with accumulation of reactive oxygen or nitrogen species comprising use of a composition comprising: i) one or more of VEGF, a VEGF biomimetic peptide, and/or a VEGFR2 receptor agonist; ii) ciclopirox olamine; and iii) a pharmaceutically acceptable carrier.

The composition of the present invention may also be used in a method for grafting follicular hair wherein the grafts are soaked in the composition prior to implantation.

Alternatively, the composition may be used in a method for implantation of follicular hair grafts wherein the composition is used after implantation to improve survival rates of hair follicles and/or to improve wound healing times.

In an additional embodiment, the present invention may be expressed as a method for hair transplant surgery comprising use of a composition comprising: i) one or more of VEGF, a VEGF biomimetic peptide, and/or a VEGFR2 receptor agonist; ii) ciclopirox olamine; and iii) a pharmaceutically acceptable carrier, wherein the composition is used to prepare follicular hair grafts for removal prior to surgery.

In a yet further additional embodiment, the present invention resides in a method for the prevention or treatment of hair loss, for improving hair growth, for improving skin blemishes, for the prevention or treatment of damage to skin cells associated with accumulation of reactive oxygen or nitrogen species, for follicular hair grafts and/or for hair transplant surgery comprising use of a composition comprising: i) one or more of VEGF, a VEGF biomimetic peptide, and/or a VEGFR2 receptor agonist; ii) ciclopirox olamine; and iii) a pharmaceutically acceptable carrier, wherein the composition is used in combination with an array of microneedles.

It will be appreciated that any of the compositions of the invention as described herein are suitable for use in the methods of treatment outlined above.

In another embodiment, the present invention resides in a kit for the treatment of hair loss, the kit comprising a) a composition comprising: i) one or more of VEGF, a VEGF biomimetic peptide, and/or a VEGFR2 receptor agonist; ii) ciclopirox olamine; and iii) a pharmaceutically acceptable carrier; and b) an array of microneedles.

It will be appreciated that any of the compositions of the invention as described herein are suitable for use in the kit outlined above.

The composition of the invention will now be demonstrated by way of non-limiting example.

Evaluation of the Activity of Ciclopirox Olamine and VEGF on Hair Growth Stimulation of Human Hairs Maintained in Survival

### 1. Explant Preparation

Isolated human hair follicles including a small sample of the surrounding skin tissue are placed individually in a 96 wells plate and maintained in survival in classical cell culture conditions (37° C., 5% CO2) with improved Williams medium over 10 days.

Ten hairs in each batch are put in a survival media and kept until it is assessed that at least seven hairs in the batch are in anagen phase. Anagen phase is assessed by the growth of the hair.

### 2. Treatment

Ciclopirox olamine, VEGF or a combination of the two is added to the culture media starting from Day 0.

Positive reference (minoxidil) and Ciclopirox olamine, VEGF or a combination of the two are tested at the following dosages:

Minoxidil 5%

Ciclopirox Olamine 0.3%, 0.5%

VEGF 1 PPM, 5PPM, 10PPM

Ciclopirox Olamine 0.3%, 0.5%+VEGF 1 PPM, 5PPM, 10PPM

Culture media is renewed every three days.

### 3. Measures and Selection of Hair in Anagen Phase (Growth Phase)

On Day-3, three days before the beginning of the treatment, all hair is cut, at about 1 mm from the infundibulum and pictures are taken with a microscope and a CCD camera coupled with a picture acquisition software. Each hair is measured with proprietary software providing measures in μm. The infundibulum is taken to be the pore around the hair shaft and, in this example, serves as a base for the measurement.

| | No. | | | Sampling |
|---|---|---|---|---|
| Batch | of hairs | Treatment | Day of measurement | day |
| B0 | 10 | None | | D0 |
| B | 10 | None | D-3, D0, D3, D6, D10 | D10 |
| PC | 10 | Positive control | D-3, D0, D3, D6, D10 | D10 |
| P1 | 10 | VEGF | D-3, D0, D3, D6, D10 | D10 |
| P2 | 10 | Ciclopirox olamine | D-3, D0, D3, D6, D10 | D10 |
| P3 | 10 | VEGF + | D-3, D0, D3, D6, D10 | D10 |
| | | Ciclopirox olamine | | |

On day 0, all hair is measured again and seven hairs in anagen phase are selected for the study. To be selected, hair must show an average growth of at least 50 μm/day over three days (between D−3 and D0).

The hair growth of each hair is followed individually in order to carry out statistical analysis at the end of the study.

On Day 3, D6 and D10, selected hairs are individually measured as described previously.

1. A composition to treat hair loss and enhance hair growth and condition, the composition comprising:
i) one or more of VEGF, a VEGF biomimetic peptide, and/or a VEGFR2 receptor agonist;
ii) ciclopirox olamine; and
iii) a pharmaceutically acceptable carrier.

2. A composition as claimed in claim 1, wherein the one or more of VEGF, a VEGF biomimetic, and/or a VEGFR2 receptor agonist is selected from the group consisting of: Copper Ascorbyl Phosphate Succinoyl Tripeptide-34; the peptide: Glycine Histadine Lysine-Cu; the peptide: R— Glycine Histadine Lysine-Cu, where R is any amino acid or peptide chain; Octapeptide-2; Decapeptide-8; Synthetic Human VEGF;
Recombinant Human VEGF; and Human VEGF from any human source.

3. A composition as claimed in claim 1, wherein ciclopirox olamine is present in an amount of between about 0.03% and about 0.50%.

4. A composition as claimed in claim 1, wherein VEGF, VEGF biomimetic, and/or VEGFR2 receptor agonist is present in an amount of between about 0.03% and about 0.12% or between about 1 and 10 ppm.

5. A composition as claimed in claim 1, wherein the composition further comprises one or more catalases, catalase mimetics, superoxide dismutases or superoxide dismutase mimetics, and wherein the superoxide dismutases are selected from the group comprising: Cu/Zn superoxide dismutase, Mn superoxide dismutase, Lipochroman-6, EUK-134, copper (II) 3,5-diisopropylsalicylate, copper (II) 3,5-dibromosalicylate, and copper (II) 3,5-ditertiarybutylsalicylate and/or wherein the catalases or catalase mimetics are selected from the group comprising: Catalase, copper PCA and zinc PCA.

6. A composition as claimed in claim 1, wherein the composition further comprises one or more antioxidants, and wherein the one or more antioxidants is selected from the group comprising: EUK-134, copper (II) 3,5-diisopropylsalicylate, copper (II) 3,5-dibromosalicylate, copper (II) 3,5-ditertiarybutylsalycilate, green tea extract and vitamin C or derivatives thereof.

7. A composition as claimed in claim 1, wherein the composition further comprises one or more 5-alpha reductase inhibitors, and wherein the one or more 5-alpha reductase inhibitors is selected from the group comprising: zinc PCA, finasteride, dutasteride, turosteride, bexlosteride, izonsteride, FCE 28260, SKF 105,111, saw palmetto extract, green tea extract and hydrolysed lupine extract.

8. A composition as claimed in claim 1, the composition further comprises one or more nitric oxide synthases, nitric oxide donators or nitric oxide mimetics, and wherein the nitric oxide synthases, nitric oxide donators or nitric oxide mimetics is selected from the group comprising: eNOS, iNOS, minoxidil, 2,2,6,6-tetramethylpiperidine-N-oxyl (TEMPO), molsidomine and S-nitrosoglutathione.

9. A composition as claimed in claim 1, wherein the composition further comprises a UV protective agent, and wherein the UV protective agent is selected from the group comprising: propanediol, quaternium-95, polyacrylate-15, titanium dioxide, zinc oxide, ethylhexyl methoxycinnamate, butyl methoxydibenzoylmethane, octyl methoxycinnamate, menthyl anthranilate, homosalate, benzophenone-3 and benzophenone-4.

10. A composition as claimed in claim 1, wherein the composition further comprises one or more cosmetic agents selected from the group consisting of: lightening agents, darkening agents, anti-acne agents, shine control agents, anti-microbial agents, anti-inflammatory agents, anti-mycotic agents, anti-parasite agents, external analgesic, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning.

11. A composition as claimed in claim 10, wherein one or more cosmetic agents are selected from the group comprising: curcumin, caffeine, saw palmetto, taurine, plant sterols, pine bark extract, red tea, white tea, horsetail extract, marine cartilage, kieslerde, melatonin and mimetics, copper peptides, other growth factors and growth factor mimetics, minoxidil, spironolactone, β-glucan, vitamins C, A, E, B, F, H, K (and derivatives), bacterial filtrates, glucosamine sulphate, and any combination thereof.

12. A composition as claimed in claim 1, wherein the composition is formulated for topical application.

13. A composition as claimed in claim 12, wherein the formulation is water-based.

14. A composition as claimed in claim 1, wherein the composition includes between about 60% to about 90% water.

15. A composition as claimed in claim 1, wherein the composition further comprises an ingredient to enhance penetration of the composition through the skin.

16. A composition as claimed in claim 21, wherein the penetration enhancing ingredient is salicylic acid.

17. A composition as claimed in claim 1, wherein one or more of ciclopirox olamine, VEGF, VEGFR2 agonists and/or VEGF biomimetic peptides are encapsulated in encapsulation vehicles such as liposomes or nanosomes.
